(19) United States
(12) Patent Application Publication
(10) Pub. No.: US 2006/0287679 A1
(43) Pub. Date: Dec. 21, 2006

(54) Method and system to control respiration by means of confounding neuro-electrical signals

(76) Inventor: Robert T. Stone, Sunnyvale, CA (US)

Correspondence Address: Ralph C. Francis;Francis Law Group, 1942 Embarcadero, Oakland, CA, 94606, US

(21) Appl. No.: 11/509,363

(22) Filed: Aug. 23, 2006

Related U.S. Application Data
(63) Continuation-in-part of application No. 11/264,937, filed on Nov. 1, 2005.
Application No. 11/264,937 is a continuation-in-part of application No. 11/129,264, filed on May 13, 2005.
Application No. 11/129,264 is a continuation-in-part of application No. 10/847,738, filed on May 17, 2004, now Pat. No. 6,937,903.
(60) Provisional application No. 60/471,104, filed on May 16, 2003.

(51) Int. Cl. A61N 1/18

(52) U.S. Cl. 607/2

(57) ABSTRACT

A method to control respiration generally comprising generating a confounding neuro-electrical signal that is adapted to confound or (suppress) at least one interneuron that induces a reflex action and transmitting the confounding neuro-electrical signal to the subject, whereby the reflex action is abated. In one embodiment, the confounding neuro-electrical signal is adapted to confound at least one parasympathetic action potential that is associated with the target reflex action, e.g., bronchial constriction.

### CROSS-REFERENCE TO RELATED APPLICATIONS

This application is a continuation-in-part of U.S. application Ser. No. 11/264,937, filed Nov. 1, 2005, which is a continuation-in-part of U.S. application Ser. No. 11/129,264, filed May 13, 2005, which is a continuation-in-part of U.S. application Ser. No. 10/847,738, filed May 17, 2004, which claims the benefit of U.S. Provisional Application No. 60/471,104, filed May 16, 2003.

### FIELD OF THE PRESENT INVENTION

The present invention relates generally to medical methods and systems for monitoring and controlling respiration. More particularly, the invention relates to a method and system for controlling respiration by means of confounding neuro-electrical signals.

### BACKGROUND OF THE INVENTION

As is well known in the art, the brain modulates (or controls) respiration via electrical signals (i.e., neurosignals or action potentials), which are transmitted through the nervous system. The nervous system includes two components: the central nervous system, which comprises the brain and the spinal cord, and the peripheral nervous system, which generally comprises groups of nerve cells (i.e., neurons) and peripheral nerves that lie outside the brain and spinal cord. The two systems are anatomically separate, but functionally interconnected.

As indicated, the peripheral nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain.

As is known in the art, a typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The cell body typically includes two types of outgrowths (or processes); the dendrites and the axon. Most neurons have several dendrites; these branch out in tree-like fashion and serve as the main apparatus for receiving signals from other nerve cells.

The axon is the main conducting unit of the neuron. The axon is capable of conveying electrical signals along distances that range from as short as 0.1 mm to as long as 2 m. Many axons split into several branches, thereby conveying information to different targets.

Near the end of the axon, the axon is divided into fine branches that make contact with other neurons. The point of contact is referred to as a synapse. The cell transmitting a signal is called the presynaptic cell, and the cell receiving the signal is referred to as the postsynaptic cell. Specialized swellings on the axon's branches (i.e., presynaptic terminals) serve as the transmitting site in the presynaptic cell.

Most axons terminate near a postsynaptic neuron's dendrites. However, communication can also occur at the cell body or, less often, at the initial segment or terminal portion of the axon of the postsynaptic cell.

Many nerves and muscles are involved in efficient respiration or breathing. The most important muscle devoted to respiration is the diaphragm. The diaphragm is a sheet-shaped muscle, which separates the thoracic cavity from the abdominal cavity.

With normal tidal breathing the diaphragm moves about 1 cm. However, in forced breathing, the diaphragm can move up to 10 cm. The left and right phrenic nerves activate diaphragm movement.

Diaphragm contraction and relaxation accounts for approximately 75% volume change in the thorax during normal quiet breathing. Contraction of the diaphragm occurs during inspiration. Expiration occurs when the diaphragm relaxes and recoils to its resting position. All movements of the diaphragm and related muscles and structures are controlled by coded electrical signals traveling from the brain.

Details of the respiratory system and related muscle structures are set forth in Co-Pending Application No. 10/847,738, which is expressly incorporated by reference herein in its entirety.

The main nerves that are involved in respiration are the ninth and tenth cranial nerves, the phrenic nerve, and the intercostal nerves. The glossopharyngeal nerve (cranial nerve IX) innervates the carotid body and senses CO₂ levels in the blood. The vagus nerve (cranial nerve X) provides sensory input from the larynx, pharynx, and thoracic viscera, including the bronchi. The phrenic nerve arises from spinal nerves C3, C4, and C5 and innervates the diaphragm. The intercostal nerves arise from spinal nerves T7-11 and innervate the intercostal muscles.

The various afferent sensory neuro-fibers provide information as to how the body should be breathing in response to events outside the body proper.

An important respiratory control is activated by the vagus nerve and its preganglionic nerve fibers, which synapse in ganglia. The ganglia are embedded in the bronchi that are also innervated with sympathetic and parasympathetic activity.

It is well documented that the sympathetic nerve division can have no effect on bronchi or it can dilate the lumen (bore) to allow more air to enter during respiration, which is helpful to asthma patients, while the parasympathetic process offers the opposite effect and can constrict the bronchi and increase secretions, which can be harmful to asthma patients.

The electrical signals transmitted along the axon to control respiration, referred to as action potentials, are rapid and transient “all-or-none” nerve impulses. Action potentials typically have an amplitude of approximately 100 millivolts (mV) and a duration of approximately 1 msec. Action potentials are conducted along the axon, without failure or distortion, at rates in the range of approximately 1-100 meters/sec. The amplitude of the action potential remains constant throughout the axon, since the impulse is continually regenerated as it traverses the axon.

A “neurosignal” is a composite signal that includes multiple action potentials. The neurosignal also includes an instruction set for proper organ function. A respiratory neurosignal would thus include an instruction set for the diaphragm to perform an efficient ventilation, including information regarding frequency, initial muscle tension, degree (or depth) of muscle movement, etc.

Neurosignals or “neuro-electrical coded signals” are thus codes that contain complete sets of information for complete organ function. As set forth herein, once these neurosignals have been isolated, a generated confounding neuro-electrical signal (i.e. suppression or masking signal) can be generated and transmitted to a subject (or patient) to mitigate various respiratory system disorders and/or one or more symptoms associated therewith. The noted disorders include, but are not limited to, asthma, acute bronchitis and emphysema.

As is known in the art, asthma is a multi-cellular redundant and self-amplifying airway disease. Asthma is typically presented by chronic inflammation of varying austerity that arises from various (genetic and environmental) etiology, e.g., innocuous environmental antigens. The pathophysiology of asthma includes mucus hyper-secretion, bronchial hyper-responsiveness, smooth muscle hypertrophy and airway constriction.

As is also well known in the art, the noted pathophysiology (or symptoms) are induced or exacerbated by respiratory neurosignals or neuro-electrical coded signals. Indeed, as indicated above, parasympathetic action potentials can induce constriction of the bronchi and increase mucus secretion.

In some instances, chronic inflammation of the lungs can be persistent even in the absence of innocuous antigens. Asthmatics can thus have airways that are hypersensitive to other environmental antigens, including viral and some bacterial infections.

On a cellular level these asthmatic symptoms arise from the activation of sub-mucosal mast cells by innocuous antigens (i.e. allergens) in the lower airways, which results in mucous and fluid accumulation, subsequently followed by bronchial constriction. The immune response to asthmatic allergens is mediated by CD4+T helper 2 (Th2) cells, eosinophils, neutrophils, macrophages, and IgE antibodies. Not surprisingly, these effector cells release cytokines that also affect expression of adhesion molecules on epithelial cells.

Without effective treatment, proinflammatory cells in a dysregulated asthmatics immune response initiate remodeling of airway tissues, commonly called subbasement membrane fibrosis. For patients with severe cases, there is a higher frequency of structural remodeling of the small airway matrix compared to patients with less severe cases; however, the later are not precluded from structural remodeling of the small airway matrix.

Asthmatic inflammation is differentiated into three broad categories: acute, subacute and chronic. Acute asthmatic inflammation involves the early recruitment of cells into the airway, while subacute asthmatic inflammation is characterized by the activation of recruited and residual effector cells resulting in incessant inflammation. Chronic asthma is defined by constant inflammation leading to cellular damage.

Asthma phenotypes are typically differentiated based upon the development of symptoms and the severity of asthmatic lung inflammation. Asthma symptoms are typically manifested at certain stages in life and can be classified into three general categories: childhood asthma, late-onset asthma and occupational asthma.

Childhood asthma can arise from several different factors. Typically, a covirial infection, such as the rhinovirus, a family history of allergy or atopy can result in the development of childhood asthma. In childhood asthma, atopy usually results from innocuous substances, such as dust mites, pet dander and fungi.

Late onset and occupational asthma exhibit different characteristics from childhood asthma and likely have a different etiology. Asthma's causation in these circumstances may arise from constant exposure to environmental innocuous antigens. The current distinction between late-onset asthma and occupational asthma is merely the fact that the latter happens usually because of specific antigen exposure related to work.

Various apparatus, systems and methods have been developed to control respiration and treat respiratory disorders, such as asthma. The systems and methods often include an apparatus for or step of recording action potentials or waveform signals that are generated in the body. The signals are, however, typically subjected to extensive processing and are subsequently employed to regulate a “mechanical” device or system, such as a ventilator. Illustrative are the systems disclosed in U.S. Pat. Nos. 6,360,740 and 6,651,652.

In U.S. Pat. No.6,360,740, a system and method for providing respiratory assistance is disclosed. The noted method includes the step of recording “breathing signals”, which are generated in the respiratory center of a patient. The “breathing signals” are processed and employed to control a muscle stimulation apparatus or ventilator.

In U.S. Pat. No. 6,651,652, a system and method for treating sleep apnea is disclosed. The noted system includes respiration sensor that is adapted to capture neuro-electrical signals and extract the signal components related to respiration. The signals are similarly processed and employed to control a ventilator.

A major drawback associated with the systems and methods disclosed in the noted patents, as well as most known systems, is that the control signals that are generated and transmitted are typically “device determinative”. The noted “control signals” are thus not related to or representative of the signals that are generated in the body and, hence, would not be operative in the control or modulation of the respiratory system if transmitted thereto.

As indicated above, in many instances the symptoms associated with asthma are induced or exacerbated by neuro-electrical coded signals, e.g., parasympathetic action potentials. Various systems and methods have thus been employed to “block” or arrest nerve conduction, i.e. block the transmission of neuro-electrical signals through a selected nerve. Illustrative are the methods disclosed in Kilgore, et al. “Nerve Conduction Block Utilising High-Frequency Alternating Current”, vol. 42, pp. 394-406, Med. Biol. Eng. Comput. (2004) and Solomonow, et al., “Control of Muscle Contractile Force Through Indirect High-Frequency Stimulation”, vol. 62, pp. 71-82, Am. Jour. of Phy. Medicine (1983).

In U.S. Pat. No. 6,684,105 and application Ser. No. 10/488,334 (Pub. No. 2004/0243182 A1) a further method for treating various disorders via nerve stimulation is disclosed. According to the disclosed methodology, low frequency (e.g., <50 Hz) signals are applied to the vagus nerve in a unidirectional mode to “block” parasympathetic action potentials, i.e. preventing the normal action potential from propagating past the point of blockage, thus preventing the triggering of the commanded effects.

There are several major drawbacks associated with the noted nerve blocking methodology. A major drawback is that the method induces a complete block of signals through a target nerve. Thus, by employing the methodology to suppress parasympathetic action potentials transmitted through the vagus nerve, the method would completely block the parasympathetic action potentials, and could, and in all likelihood would, block additional natural biologic action potentials that are essential to regulate the respiratory system.

A further drawback is that, in many instances, the stimulus levels that are required to achieve the nerve block are excessive and can elicit deleterious side effects.

It would thus be desirable to provide a method and system for controlling respiration that includes means for generating and transmitting confounding neuro-electrical signals to the body that are adapted to confound (or suppress) neurosignals (or action potentials) that are generated in the body and are associated with symptoms of a respiratory disorder, such as bronchial constriction, whereby the symptom (or symptoms) are abated.

It is therefore an object of the present invention to provide a method and system for controlling respiration that overcomes the drawbacks associated with prior art methods and systems for controlling respiration.

It is another object of the invention to provide a method and system for controlling respiration that includes means for generating and transmitting confounding neuro-electrical signals to the body that are adapted to confound (or suppress) neurosignals (or action potentials) that are generated in the body and are associated with symptoms of a respiratory disorder, whereby a symptom (or symptoms) is abated.

It is another object of the invention to provide a method and system for controlling respiration that includes means for generating and transmitting confounding neuro-electrical signals to the body that are adapted to confound parasympathetic action potentials that are generated in the body.

It is another object of the invention to provide a method and system for treating bronchial constriction by generating and transmitting confounding neuro-electrical signals to the vagus nerve that are adapted to confound parasympathetic action potentials that are associated with bronchial constriction.

It is another object of the invention to provide a method and system for controlling respiration that includes means for generating simulated action potential signals that substantially correspond to coded waveform signals that are generated in the body and are operative in the control of respiratory system.

It is another object of the present invention to provide a method for generating simulated action potential signals that is based on a digital approximation of coded waveform signals that are generated in the body.

It is another object of the invention to provide a method and system for generating confounding neuro-electrical signals based on the generated simulated action potential signals.

It is another object of the invention to provide a method and system for controlling respiration that includes means for recording waveform signals that are generated in the body and operative in the control of respiration.

It is another object of the invention to provide a method and system for controlling respiration that includes processing means adapted to generate a base-line respiratory signal that is representative of at least one coded waveform signal generated in the body from recorded waveform signals.

It is another object of the invention to provide a method and system for controlling respiration that includes processing means adapted to compare recorded respiratory waveform signals to baseline respiratory signals and generate a respiratory signal as a function of the recorded waveform signal.

It is another object of the invention to provide a method and system for controlling respiration that includes monitoring means for detecting respiration abnormalities.

It is another object of the invention to provide a method and system for controlling respiration that includes a sensor to detect whether a subject is experiencing a respiratory disorder.

It is another object of the invention to provide a method and system for controlling respiration that can be readily employed in the treatment of respiratory system disorders, including, asthma, excessive mucus production, acute bronchitis and emphysema.

### SUMMARY OF THE INVENTION

In accordance with the above objects and those that will be mentioned and will become apparent below, the method to control respiration, in one embodiment, generally includes the steps of (i) generating a confounding neuro-electrical signal that is adapted to confound or (suppress) at least one interneuron that induces a reflex action, and (ii) transmitting the confounding neuro-electrical signal to the subject, whereby the interneuron is suppressed.

In one embodiment, the confounding neuro-electrical signal is adapted to suppress at least one parasympathetic action potential that is associated with the target reflex action, e.g., bronchial constriction.

In accordance with another embodiment of the invention, there is also provided a method for treating (or inhibiting) bronchial constriction of a subject that includes the steps of (i) generating a confounding neuro-electrical signal that is adapted to confound or (suppress) at least one group of reflex mediating interneurons that induces bronchial constriction, and (ii) transmitting the confounding neuro-electrical signal to the subject, whereby bronchial constriction is abated.

In one embodiment of the invention, the confounding neuro-electrical signal includes a plurality of simulated action potential signals, the simulated action potential signals having a first region having a positive voltage in the range of approximately 100-2000 mV for a first period of time in the range of approximately 100-400 μsec and a second region having a negative voltage in the range of approximately −50 mV to −1000 mV for a second period of time in the range of approximately 200-800 μsec.

In one embodiment, the confounding neuro-electrical signal has a frequency in the range of approximately 1-2 KHz.

In accordance with a further embodiment, there is provided a method for treating a pathophysiology of asthma in a subject that includes the steps of (i) generating a confounding neuro-electrical signal that is adapted to suppress at least one abnormal respiratory signal that induces a pathophysiology of asthma, and (ii) transmitting the confounding neuro-electrical signal to the nervous system of the subject, whereby the pathophysiology is abated.

In one embodiment, the pathophysiology is selected from the group consisting of bronchial hyper-responsiveness, smooth muscle hypertrophy, mucus hyper-secretion and hyper-secretion of a proinflammatory cytokine.

In another embodiment of the invention, the method to control respiration generally includes the steps of (i) generating a confounding neuro-electrical signal, the confounding neuro-electrical signal including a plurality of simulated action potential signals, the simulated action potential signals having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec, and (ii) transmitting the confounding neuro-electrical signal to the body to control the respiratory system.

In one embodiment, the confounding neuro-electrical signal has a frequency in the range of approximately 1-2 KHz.

In another embodiment of the invention, the method to control respiration generally includes the steps of (i) generating a simulated action potential signal having a first region having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec, (ii) generating a confounding neuro-electrical signal, the confounding neuro-electrical signal including a plurality of the simulated action potential signals, and (iii) transmitting the confounding neuro-electrical signal to the body to control the respiratory system.

In one embodiment, the confounding neuro-electrical signal has a frequency in the range of approximately 1-2 KHz.

In another embodiment, the method to control respiration generally includes the steps of (i) generating a random confounding neuro-electrical signal, the random confounding neuro-electrical signal including a plurality of random simulated action potential signals, the random simulated action potential signals having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec, and (ii) transmitting the random confounding neuro-electrical signal to the body to control the respiratory system.

In one embodiment, the random confounding neuro-electrical signal has a frequency in the range of approximately 1-2 KHz.

According to the invention, the random simulated action potential signals have randomly varied positive amplitude and/or first period of time and/or negative amplitude and/or second period of time.

In one embodiment, the random confounding neuro-electrical signal has a randomly varied frequency.

In another embodiment of the invention, the method to control respiration generally includes the steps of generating a pseudo-random confounding neuro-electrical signal, the pseudo-random confounding neuro-electrical signal including a plurality of pseudo-random simulated action potential signals, the pseudo-random simulated action potential signals similarly having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec, and (ii) transmitting the pseudo-random confounding neuro-electrical signal to the body to control the respiratory system.

In one embodiment, the pseudo-random confounding neuro-electrical signal has a frequency in the range of approximately 1-2 KHz.

According to the invention, the pseudo-random simulated action potential signals have pseudo-randomly varied positive amplitude and/or first period of time and/or negative amplitude and/or second period of time.

In one embodiment, the pseudo-random confounding neuro-electrical signal has a pseudo-randomly varied frequency.

In accordance with a further embodiment of the invention, the method for controlling respiration in a subject generally includes the steps of (i) generating a steady state, random or pseudo-random confounding neuro-electrical signal, (ii) monitoring the respiration status of the subject and providing at least one respiratory system status signal in response to an abnormal function of the respiratory system, and (iii) transmitting the steady state, random or pseudo-random confounding neuro-electrical signal to the body in response to a respiratory status signal that is indicative of respiratory distress or a respiratory abnormality.

Preferably, the generated confounding neuro-electrical signals are transmitted to the vagus nerve of a subject.

In accordance with a further embodiment of the invention, there is provided a confounding neuro-electrical signal, the confounding neuro-electrical signal including a plurality of simulated action potential signals, the simulated action potential signals having a first region having a positive amplitude in the range of approximately 100-2000 mV for a first period of time in the range of approximately 100-400 μsec, a second region having a negative amplitude in the range of approximately −50 mV to −1000 mV for a second period of time in the range of approximately 200-800 μsec and a frequency in the range of approximately 0.5-4 KHz, the confounding neuro-electrical signal being adapted to suppress at least one interneuron that induces a reflex action in the body when transmitted thereto.

In one embodiment, the confounding neuro-electrical signal has a frequency in the range of approximately 1-2 KHz.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A and 1B are illustrations of transmitted waveform signals (neurosignals) captured from the phrenic nerve of a mammal that are operative in the control of the respiratory system;

FIG. 2 is a schematic illustration of one embodiment of a simulated action potential signal that has been generated by the process means of the invention;

FIG. 3A is a further illustration of a transmitted waveform signal that is operative in the control of the respiratory system;

FIG. 3B is an illustration of the transmitted waveform signal shown in FIG. 3A and a simultaneously transmitted confounding neuro-electrical signal, illustrating the suppression or masking of the waveform signal according to the invention;

FIGS. 4 and 5 are illustrations of waveform signals captured from the phrenic nerve of a rat;

FIG. 6 is a graphical illustration of the frequency distribution of the waveform signal shown in FIG. 4;

FIG. 7 is a schematic illustration of one embodiment of a respiratory control system, according to the invention;

FIG. 8 is a schematic illustration of another embodiment of a respiratory control system, according to the invention;

FIG. 9 is a schematic illustration of yet another embodiment of a respiratory control system, according to the invention;

FIG. 10 is a schematic illustration of an embodiment of a respiratory control system that can be employed in the treatment of a respiratory disorder, according to the invention;

FIG. 11 is a graphical illustration of arterial saturation during a methacholine challenge with and without the administration of a confounding neuro-electrical signal; and

FIG. 12 is a graphical illustration of partial pressure of arterial oxygen during a methacholine challenge with and without the administration of a confounding neuro-electrical signal.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a confounding neuro-electrical signal” includes two or more such signals; reference to “a respiratory disorder” includes two or more such disorders and the like.

### Definitions

The term “respiratory system”, as used herein, means and includes, without limitation, the organs subserving the function of respiration, including the diaphragm, lungs, nose, throat, larynx, trachea and bronchi, and the nervous system associated therewith.

The term “respiration”, as used herein, means the process of breathing.

The terms “respiratory system disorder”, “respiratory disorder” and “adverse respiratory event”, as used herein, mean and include any dysfunction of the respiratory system that impedes the normal respiration process. Such dysfunction can be presented or caused by a multitude of known factors and events, including, mucus hyper-sccretion, bronchial hyper-responsiveness, smooth muscle hypertrophy and airway constriction or obstruction.

The term “asthma”, as used herein, means and includes a respiratory system disorder that is characterized by at least one of the following: smooth muscle hypertrophy, airway constriction or obstruction, mucus hyper-secretion or bronchial hyper-responsiveness.

The term “nervous system”, as used herein, means and includes the central nervous system, including the spinal cord, medulla, pons, cerebellum, midbrain, diencephalon and cerebral hemisphere, and the peripheral nervous system, including the neurons and glia.

The term “plexus”, as used herein, means and includes a branching or tangle of nerve fibers outside the central nervous system.

The term “ganglion”, as used herein, means and includes a group or groups of nerve cell bodies located outside the central nervous system.

The terms “waveform” and “waveform signal”, as used herein, mean and include a composite electrical signal that is naturally generated in the body (humans and animals) and carried by neurons in the body, including action potentials, neurocodes, neurosignals and components and segments thereof.

The term “pseudo-random”, as used herein in connection with “confounding neuro-electrical signals”, means a generated neuro-electrical signal and/or train thereof having a pre-determined or computed variation in amplitude, frequency of occurrence, period (or frequency segment), interval(s) between signals or any combination thereof.

The term “random”, as used herein in connection with “confounding neuro-electrical signals”, means a generated neuro-electrical signal and/or train thereof having a variation in amplitude, frequency of occurrence, period (or frequency segment), interval(s) between signals or any combination thereof, whereby the amount of variation is determined by a truly random event, such as thermal noise in an electronic component.

The term “sympathetic action potential”, as used herein, means a neuro-electrical signal that is transmitted through sympathetic fibers of the automic nervous system and tends to depress secretion, and decrease the tone and contractility of smooth muscle, e.g., bronchial dilation.

The term “parasympathetic action potential”, as used herein, means a neuro-electrical signal that is transmitted through parasympathetic fibers of the automic nervous system and tends to induce secretion and increase the tone and contractility of smooth muscle, e.g., bronchial constriction.

The term “abnormal respiratory signal”, as used herein, means and includes an electrical signal (i.e. respiratory neurosignal) or component thereof that induces a pathophysiology (or symptom) of asthma, including, without limitation, bronchial hyper-responsiveness (or constriction), smooth muscle hypertrophy, mucus hyper-secretion and hyper-secretion of a proinflammatory cytokine. The term “abnormal respiratory signal” can thus include “parasympathetic action potentials”.

The term “simulated action potential signal”, as used herein, means and includes a generated neuro-electrical signal that is operative in the regulation of body organ function, including the respiratory system. In one embodiment of the invention, the “simulated action potential signal” comprises a biphasic signal that exhibits positive voltage (or current) for a first period of time and negative voltage for a second period of time. The term “simulated action potential signal” thus includes square wave signals, modified square wave signals and frequency modulated signals.

In one embodiment of the invention, the “simulated action potential signal” comprises a neuro-electrical signal or component thereof that substantially corresponds to a “waveform signal”.

The terms “confound”, “over-ride”, “confuse”, “suppress” and “mask”, as used herein in connection with a waveform signal and/or neuro-electrical signal and/or action potential (e.g., sympathetic and parasympathetic action potentials), mean diminishing the effectiveness of interneurons that normally induce a reflex action or causing such interneurons to be ignored by the body.

The term “confounding neuro-electrical signal”, as used herein, means and includes an electrical signal that mimics either sensory or effector signals on the nerve, whereby the interneurons that are normally active in interpretation and effecting of reflex actions do not effect the expected reflex. A “confounding neuro-electrical signal” can thus comprise an “over-riding signal” or a signal that confounds or confuses the interneuron, whereby the target effector signal(s) are suppressed.

The term “signal train”, as used herein, means a composite signal having a plurality of signals, such as the “simulated action potential signal” and “confounding neuro-electrical signal” defined above.

Unless stated otherwise, the confounding neuro-electrical signals of the invention are designed and adapted to be transmitted continuously or at set, i.e. predetermined steady state or variable, intervals to a subject.

The term “target zone”, as used herein, means and includes, without limitation, a region of the body proximal to a portion of the nervous system whereon the application of electrical signals can induce the desired neural control without the direct application (or conduction) of the signals to a target nerve.

The terms “patient” and “subject”, as used herein, mean and include humans and animals.

The present invention substantially reduces or eliminates the disadvantages and drawbacks associated with prior art methods and systems for controlling respiration. As will be readily apparent to one having ordinary skill in the art, the methods and systems of the invention, described in detail below, can be readily and effectively employed in the treatment of a multitude of respiratory disorders; particularly, asthma.

As indicated above, asthma is a respiratory disorder that is characterized by two primary and distinct symptoms. The first symptom is a constriction of the airways due to contraction of the smooth muscle tissue lining the bronchi and bronchioles. This is believed to be due to hyper-reactive reflex triggered by sensory nerves lining the bronchi.

As is known in the art, the sensory nerve signals trigger reflex loops that are locally mediated by interneurons in the ganglia located in the vagus nerve which innervates the lung, and a larger reflex loop mediated by interneurons located in the brainstem. This hyper-reactive reflex causes constriction and mucus secretion that inhibits normal respiration, and can be so severe as to be life-threatening.

The second asthma symptom is characterized by inflammation of the airways, which may be similarly triggered by the noted sensory nerve signal trigger(s) or by allergic reactions from inhaled agents, or as a result of respiratory infections.

As discussed in detail below, the methods and systems of the invention are directed to alleviating respiratory disorders and/or the symptoms associated therewith in a subject; particularly, the symptoms associated with asthma by transmitting a confounding neuro-electrical signal to the subject that is adapted to over-ride or suppress or confuse interneurons that are normally active in interpretation and effecting of reflex actions, whereby they do not effect the expected reflex. In one embodiment of the invention, the confounding neuro-electrical signal is adapted to confound at least one parasympathetic effector signal that is associated with the target reflex action, e.g., bronchial constriction.

Thus, in one embodiment of the invention, the method for controlling respiration in a subject includes the steps of generating a confounding neuro-electrical signal that is adapted to confound or (suppress) at least one interneuron that induces a reflex action, and (ii) transmitting the confounding neuro-electrical signal to the subject, whereby the interneuron is suppressed.

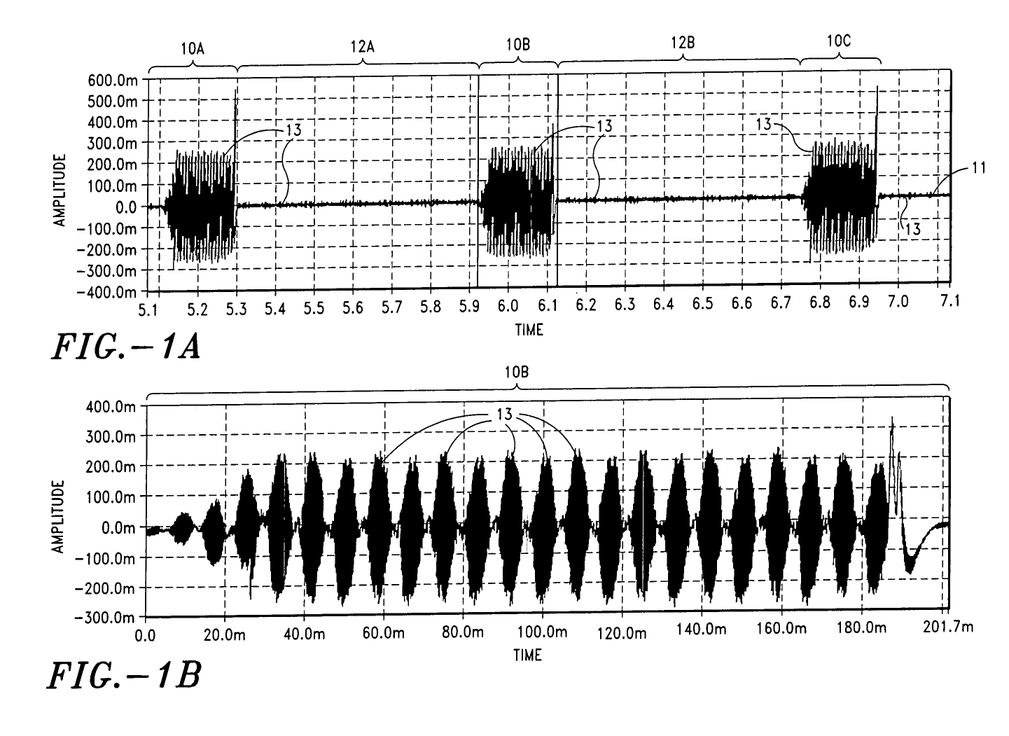

Referring now to FIGS. 1A and 1B, there is shown an exemplar waveform signal (or neurosignal) 11 that is operative in the efferent operation of the human (and animal) diaphragm; FIG. 1A showing three (3) signal bursts or segments 10A, 10B, 10C, having intervals, i.e. 12A, 12B, therebetween, and FIG. 1B showing an expanded view of signal segment 10B. As is well known in the art, the intervals can comprise a region of lower intensity (or amplitude) action potentials and/or frequency. The noted signal traverses the phrenic nerve, which runs between the cervical spine and the diaphragm.

As stated above, the signal 11 includes coded information related to inspiration, such as frequency, initial muscle tension, degree (or depth) of muscle movement, etc. The signal also includes coded information related to (i.e. controls) various sympathetic and parasympathetic actions, including bronchial constriction and mucus secretion.

As illustrated in FIG. 1B, signal segment 10B (as well as signal segments 10A, 10C and intervals 12A, 12B) comprises a plurality of action potentials 13. As is well known in the art, the net intensity of a neurosignal that effects an action (e.g., muscle contraction) is a function of the number of action potentials that are transmitted to the target muscle. The carrier intensity is thus the frequency of the signal.

The coded information that is included in and, hence, transmitted by a neurosignal, i.e. a plurality of action potentials, is embodied in or a function of the modulation of the frequency. Thus, to read or interpret the coded information, the target organ or system must be able to read the modulation of the frequency over the entire neurosignal, including the intervals between signal segments or bursts, e.g., 12A and 12B.

As is also known in the art, waveform signals or biologic action potentials typically exhibit an exponential rise from zero to 100 mV; followed by an exponential decay to a negative voltage of approximately −35 mV; followed by a gradual return to zero voltage; all of which occurring over an interval of approximately 1 millisecond.

The neuron is unable to produce another action potential until the negative voltage has returned near a baseline of zero voltage. Thus, the maximum rate at which a single neuron is capable of firing is somewhere between 1000 and 2000 times per second.

Thus, in one embodiment of the invention, a digital approximation of an action potential is employed to generate a simulated action potential. According to the invention, the first portion of the approximation comprises a positive, preferably rectangular voltage (or current ) pulse of sufficient amplitude to trigger depolarization of axon membranes near the electrode, which is preferably immediately followed by a second portion comprising negative voltage (or current) that is sufficient to facilitate repolarization of the axons near the stimulating electrode. The durations of the positive and negative portions of the digital approximation are always of the same order of magnitude as biologic action potentials, i.e. 0.5-1.5 milliseconds.

Applicant has found that the use of the noted digital approximation of an action potential and low amplitude stimulation prevent saturation or blocking of the nerve, while allowing the introduction of either enabling commands based on prior recordings or confounding neuro-electrical signals (discussed below) that suppress and/or disable the prior recoded signals.

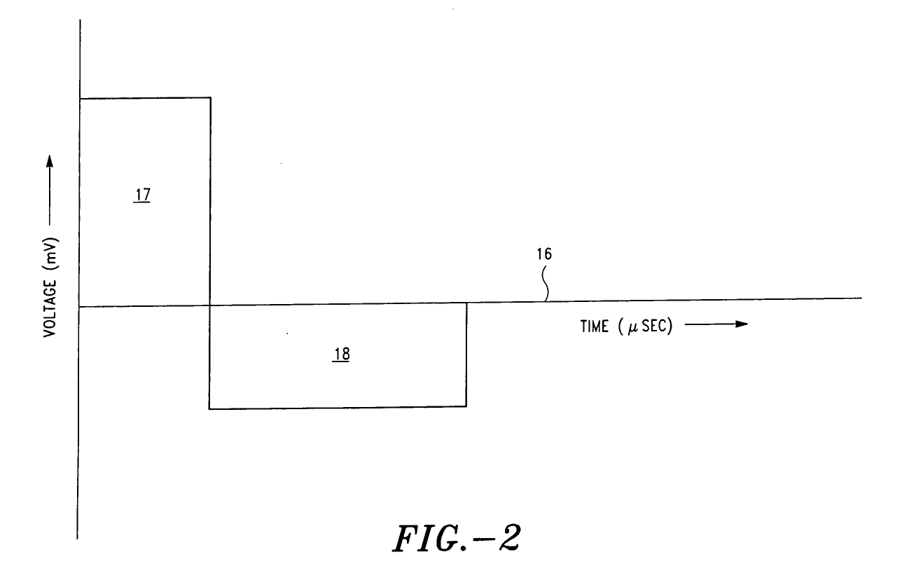

Referring now to FIG. 2, there is shown one embodiment of a simulated action potential signal 16 of the invention. As illustrated in FIG. 2, the simulated action potential signal 16 comprises a modified, substantially square wave signal. According to the invention, the simulated action potential signal 16 includes a positive voltage region 17 having a positive voltage (V₁) for a first period of time (T₁) and a negative voltage region 18 having a negative voltage (V₂) for a second period of time (T₂).

In a preferred embodiment of the invention, the positive voltage (V₁) is in the range of approximately 100 to 2000 mV, more preferably, in the range of approximately 700 to 900 mV, even more preferably, approximately 800 mV; the first period of time (T₁) is in the range of approximately 100 to 400 μsec, more preferably, in the range of approximately 150 to 300 μsec, even more preferably, approximately 200 μsec; the negative voltage (V₂) is in the range of approximately −50 mV to −1000 mV, more preferably, in the range of approximately −350 mV to −450 mV, even more preferably, approximately −400 mV; the second period of time (T₂) is in the range of approximately 200 to 800 μsec, more preferably, in the range of approximately 300 to 600 μsec, even more preferably, approximately 400 μsec.

As will be appreciated by one having ordinary skill in the art, the effective amplitude for the applied voltage is a strong function of several factors, including the electrode employed, the placement of the electrode and the preparation of the nerve.

The simulated action potential signal 16 thus comprises a biphasic signal, i.e. a substantially continuous sequence (or bursts) of positive and negative substantially square waves of voltage (or current), which preferably exhibits a DC component signal substantially equal to zero.

In a preferred embodiment of the invention, the simulated action potential signal 16 substantially corresponds to or is representative of an action potential signal that is naturally generated in a body (human and/or animal).

According to the invention, the simulated action potential signals of the invention are employed to generate the confounding neuro-electrical signals of the invention. Thus, in one embodiment of the invention, the confounding neuro-electrical signal comprises a plurality of simulated action potential signals.

Preferably, the confounding neuro-electrical signal has a repetition rate or frequency in the range of approximately 0.5-4 KHz, more preferably, in the range of approximately 1-2 KHz. Even more preferably, the frequency is approximately 1.6 KHz.

As will be readily apparent by one having ordinary skill in the art, in some instances, the generated confounding neuro-electrical signals can correspond to at least one neurosignal (or waveform signal) that is naturally generated in the body.

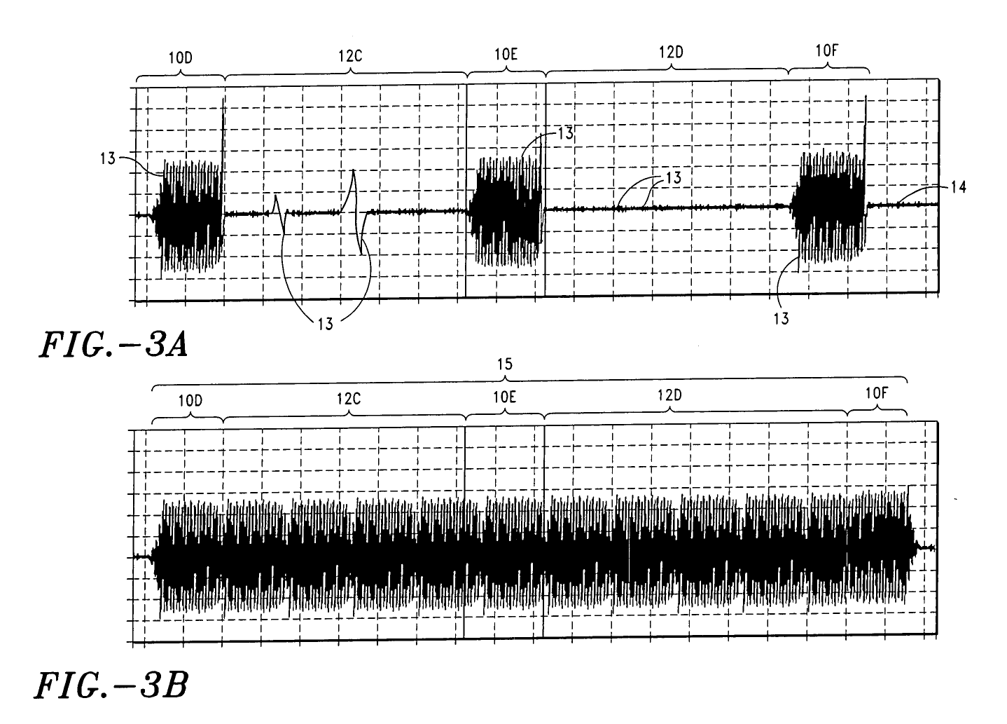

According to the invention, when a confounding neuro-electrical signal of the invention is transmitted to a target nerve, e.g., vagus nerve, the confounding neuro-electrical signal mimics the sensory of effector signal (or signals) on the nerve, whereby the signal(s) are suppressed or masked. This phenomenon is illustrated in FIGS. 3A and 3B.

Referring first to FIG. 3A, there is shown an exemplar neurosignal 14. The neurosignal 14 comprises signal segments 10D, 10E and 10F, and intervals 12C and 12D. As illustrated in FIG. 3A, each segment 10D-10F and interval 12C, 12D includes a plurality of action potentials 13.

Referring now to FIG. 3B, there is shown an illustration of signal 14 and a confounding neuro-electrical signal 15 that is simultaneously transmitted therewith. As will be appreciated by one having ordinary skill in the art, although signal 14 is still being transmitted, the body, i.e. target organ, cannot read or interpret the coded information embodied in the signal intervals 12C, 12D, since the target organ cannot read the modulation of the frequency therein. The signal 14 is thus suppressed or masked and, hence, cannot effect a reflex action.

Applicant has also determined that naturally generated action potentials that traverse a nerve body typically exhibit variable parameters, such as amplitude and frequency. The noted phenomenon is illustrated in FIGS. 4-6.

Referring first to FIG. 4, there is shown an illustration of a neurosignal (or waveform signal) 19 obtained from the phrenic nerve of a rat during spontaneous inspiration. The data acquisition rate was approximately 50 KHz, whereby the time period of the illustrated signal 19 is approximately 0.5 seconds.

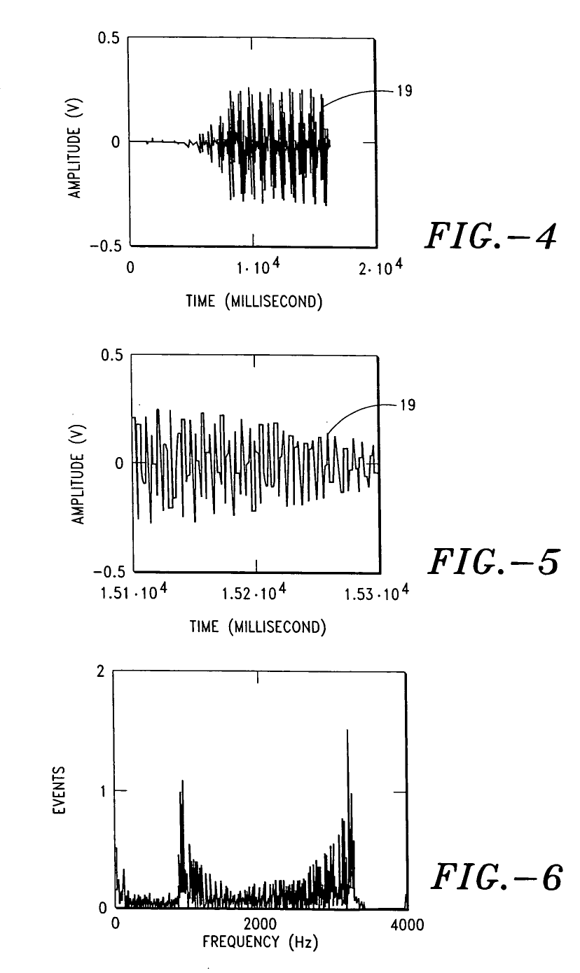

FIG. 5 is an expanded view of signal 19, representing an interval of approximately 5.0 milliseconds.

Referring now to FIG. 6, there is shown the frequency distribution (or content) of the neurosignal 19 shown in FIG. 4. It can be seen that signal 19 exhibits virtually all frequencies from approximately 500 Hz to over 3000 Hz. This establishes the presence of multiple pulsatile events, which occur at irregular intervals, i.e. random or pseudo-random intervals between signals.

Thus, in one embodiment of the invention, the confounding neuro-electrical signal comprises a random confounding neuro-electrical signal having a plurality of “random simulated action potential signals”. According to the invention, the random simulated action potential signals can have randomly varied positive voltage (V₁) or amplitude and/or first period of time (T₁) and/or negative voltage (V₂) or amplitude and/or second period of time (T₂).

The random confounding neuro-electrical signal can also have randomly varied frequency and/or intervals or rest periods between signals.

Thus, in one embodiment, the random simulated action potential signal comprises a simulated action potential signal having a randomly varied positive amplitude (V₁). In another embodiment, the random simulated action potential signal comprises a simulated action potential signal having a randomly varied negative amplitude (V₂). In another embodiment, the random simulated action potential signal comprises a simulated action potential signal having a randomly varied first period of time (T₁). In another embodiment, the random simulated action potential signal comprises a simulated action potential signal having a randomly varied second period of time (T₂).

Preferably, the normalized positive amplitude of the random simulated action potential signal is randomly varied between approximately 0.5-1.5, more preferably, between approximately 0.95-1.05 times the average positive amplitude. Preferably, the normalized negative amplitude is similarly randomly varied between approximately 0.5-1.5, more preferably, between approximately 0.95-1.05 times the average negative amplitude.

Preferably, the periods (T₁) and (T₂) of the random simulated action potential signal are randomly varied between approximately 0.25-5.0 milliseconds, more preferably, between 0.5-1.0 millisecond.

Preferably, the frequency of the random confounding neuro-electrical signal is randomly varied between approximately 40-4000 Hz, more preferably, between approximately 1000-2000 Hz.

As will be appreciated by one having ordinary skill in the art, the noted random variations in amplitude, period, frequency and signal intervals (including the signal train intervals, discussed below) can be determined by a random noise generator incorporated in the circuitry of the control systems described herein.

In another embodiment of the invention, the confounding neuro-electrical signal comprises a pseudo-random confounding neuro-electrical signal having a plurality of “pseudo-random simulated action potential signals”. According to the invention, the pseudo-random simulated action potential signals can have pseudo-randomly varied positive voltage (V₁) or amplitude and/or first period of time (T₁) and/or negative voltage (V₂) or amplitude and/or second period of time (T₂).

The pseudo-random confounding neuro-electrical signal can also have a pseudo-randomly varied frequency and/or intervals or rest periods between signals.

Thus, in one embodiment, the pseudo-random simulated action potential signal comprises a simulated action potential signal having a pseudo-random variations in positive amplitude (V₁). In another embodiment, the pseudo-random simulated action potential signal comprises a simulated action potential signal having pseudo-random variations in negative amplitude (V₂). In another embodiment, the pseudo-random simulated action potential signal comprises a simulated action potential signal having pseudo-random variations in the first period of time (T₁). In another embodiment, the pseudo-random simulated action potential signal comprises a simulated action potential signal having pseudo-random variations in the second period of time (T₂).

Preferably, the normalized positive amplitude of the pseudo-random simulated action potential signal is pseudo-randomly varied between approximately 0.5-1.5 times the average positive amplitude, more preferably, between approximately 0.95-1.05 times the average positive amplitude. Preferably, the normalized negative amplitude of the pseudo-random simulated action potential signal is similarly pseudo-randomly varied between approximately 0.5-1.5, more preferably, between approximately 0.95-1.05 times the average negative amplitude.

Preferably, the periods (T₁) and (T₂) are pseudo-randomly varied between approximately 0.25-5.0 milliseconds, more preferably, between approximately 0.5-1.0 millisecond.

Preferably, the frequency of the pseudo-random confounding neuro-electrical signal is pseudo-randomly varied between approximately 40-4000 Hz, more preferably, between approximately 1000-2000 Hz.

As will be appreciated by one having ordinary skill in the art, the noted pseudo-random variations in amplitude, period, frequency and signal intervals (including the signal train intervals, discussed below) can be determined by a pseudo-random noise generator incorporated in the circuitry of the control systems described herein.

As indicated, the steady state, random and pseudo-random simulated action potential signals are employed to construct the steady state, random and pseudo-random confounding neuro-electrical signals or “signal trains” of the invention, which comprise a plurality of steady state simulated action potential signals and/or random simulated action potential signals and/or pseudo-random simulated action potential signals. According to the invention, the noted confounding neuro-electrical signals or signal trains can include substantially uniform, randomly varied and/or pseudo-randomly varied interposed rest periods, e.g., zero voltage and current, between the simulated action potential signals and/or random simulated action potential signals and/or pseudo-random simulated action potential signals. The signal trains can also include one or more regions of lower amplitude and/or frequency signal segments (i.e., action potentials) and/or interposed supplemental signals.

Thus, in one embodiment, there is provided a random confounding neuro-electrical signal comprising a plurality of simulated action potential signals having randomly varied intervals (i.e. rest periods) therebetween. In another embodiment, the random confounding neuro-electrical signal comprises a plurality of random simulated action potential signals having randomly varied intervals (i.e. rest periods) therebetween.

Preferably, the interval between the simulated action potential signals (and random simulated action potential signals) is randomly varied between approximately 0.25-5.0 milliseconds, more preferably, between approximately 0.5-1.0 millisecond.

In yet another embodiment, the random confounding neuro-electrical signal comprises a plurality of random confounding neuro-electrical signals having substantially uniform or random intervals therebetween. As will be appreciated by one having ordinary skill in the art, the interval(s) between the random confounding neuro-electrical signals can be a few milliseconds to several seconds, e.g., 0.3 millisecond-10 seconds. In one embodiment of the invention, the interval between the random confounding neuro-electrical signals is in the range of approximately 0.4-2.0 milliseconds, more preferably, in the range of approximately 0.5-0.8 millisecond.

In accordance with at least one embodiment of the invention, the interval between the random confounding neuro-electrical signals is preferably randomly varied between approximately 0.4-2.0 milliseconds. More preferably, the interval between the random confounding neuro-electrical signals is preferably randomly varied between approximately 0.5-0.8 millisecond.

In another embodiment, there is provided a pseudo-random confounding neuro-electrical signal comprising a plurality of simulated action potential signals having pseudo-random variations in the intervals between signals (i.e. rest periods). In another embodiment, the pseudo-random confounding neuro-electrical signal comprises a plurality of pseudo-random simulated action potential signals having pseudo-random variations in the intervals between signals.

Preferably, the interval between the simulated action potential signals (and pseudo-random simulated action potential signals) is pseudo-randomly varied between approximately 0.25-5.0 milliseconds, more preferably, between approximately 0.5-1.0 millisecond.

In yet another embodiment, the pseudo-random confounding neuro-electrical signal comprises a plurality of pseudo-random confounding neuro-electrical signals having substantially uniform or pseudo-random intervals therebetween. According to the invention, the interval between the pseudo-random confounding neuro-electrical signals is similarly in the range of approximately 0.002-0.33 second, more preferably, in the range of approximately 0.008-0.01 second. In one embodiment of the invention, the interval between the pseudo-random confounding neuro-electrical signals is preferably pseudo-randomly varied between approximately 0.002-0.2 second, more preferably, between approximately 0.005-0.01 second.

Hereinafter, unless expressly stated otherwise, the term confounding neuro-electrical signal includes steady state, random and pseudo-random confounding neuro-electrical signals.

In some embodiments of the invention, the methods for controlling respiration in a subject include the step of capturing neurosignals (or waveform signals) from a subject's body that are operative in the regulation of the respiratory system. According to the invention, the captured neurosignals can be employed to generate simulated action potential signals.

As indicated, neurosignals related to respiration (i.e. respiratory neurosignals) originate in the respiratory center of the medulla oblongata. These signals can be captured or collected from the respiratory center or along the nerves carrying the signals to the respiratory musculature. The phrenic nerve has, however, proved particularly suitable for capturing the noted signals.

Methods and systems for capturing respiratory neurosignals from the phrenic nerve(s), and for storing, processing and transmitting neuro-electrical signals (or coded waveform signals) are set forth in Co-Pending application Ser. Nos. 10/000,005, filed Nov. 20, 2001, and application Ser. No. 11/125,480 filed May 9, 2005; which are incorporated by reference herein in their entirety.

According to one embodiment of the invention, the captured neurosignals are preferably transmitted to a processor or control module. Preferably, the control module includes storage means adapted to store the captured signals. In a preferred embodiment, the control module is further adapted to store the components of the captured signals (that are extracted by the processor) in the storage means according to the function performed by the signal components.

As indicated, according to one embodiment of the invention, the captured neurosignals are processed by known means to generate a simulated action potential signal of the invention. In a preferred embodiment, the simulated action potential signal substantially corresponds to or is representative of at least one signal segment (i.e. action potential) of a captured neurosignal. The generated simulated action potential signal is similarly preferably stored in the storage means of the control module.

As indicated above, the generated simulated action potential signals are employed to construct the confounding neuro-electrical signals of the invention. The confounding neuro-electrical signals are similarly preferably stored in the storage means of the control module.

According to the invention, the stored neurosignals can also be employed to establish base-line respiratory signals. The module can then be programmed to compare neurosignals (and components thereof) captured from a subject to base-line respiratory signals and generate a neuro-electrical signal or simulated action potential signal based on the comparison for transmission to a subject.

In accordance with one embodiment of the invention, the confounding neuro-electrical signal is accessed from the storage means and transmitted to the subject via a transmitter (or probe) to control respiration, e.g., abate bronchial constriction.

Thus, the method for controlling respiration in a subject, in one embodiment, includes the steps of (i) generating a simulated action potential signal having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec, (ii) generating a confounding neuro-electrical signal, the confounding neuro-electrical signal including a plurality of the simulated action potential signals, and (iii) transmitting the confounding neuro-electrical signal to the body to control the respiratory system.

In one embodiment, the confounding neuro-electrical signal has a frequency in the range of approximately 0.5-4 KHz.

In another embodiment, the confounding neuro-electrical signal has a frequency in the range of approximately 1-2 KHz.

In another embodiment, the method for controlling respiration in a subject includes the steps of (i) generating a confounding neuro-electrical signal that is adapted to confound or (suppress) at least one interneuron that induces a reflex action (associated with an asthma symptom) and (ii) transmitting the confounding neuro-electrical signal to the subject. In one embodiment, the confounding neuro-electrical signal is adapted to confound at least one parasympathetic action potential that is associated with the target reflex action, e.g., bronchial constriction.

In accordance with another embodiment of the invention, there is also provided a method for treating (or inhibiting) bronchial constriction of a subject that similarly includes the steps of (i) generating a confounding neuro-electrical signal that is adapted to confound or (suppress) at least one group of reflex mediating interneurons that induces bronchial constriction and (ii) transmitting the confounding neuro-electrical signal to the subject, whereby bronchial constriction is abated.

In accordance with a further embodiment, there is provided a method for treating a pathophysiology of asthma in a subject that includes the steps of (i) generating a confounding neuro-electrical signal that is adapted to suppress at least one abnormal respiratory signal that induces a pathophysiology of asthma, and (ii) transmitting the confounding neuro-electrical signal to the nervous system of the subject, whereby the pathophysiology is abated.

In one embodiment, the pathophysiology is selected from the group consisting of bronchial hyper-responsiveness, smooth muscle hypertrophy, mucus hyper-secretion and hyper-secretion of a proinflammatory cytokine.

In another embodiment, the method to control respiration generally includes the steps of (i) generating a steady state, random or pseudo-random confounding neuro-electrical signal, the steady state, random or pseudo-random confounding neuro-electrical signal including a plurality of random simulated action potential signals, the random simulated action potential signals having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec, and (ii) transmitting the steady state, random or pseudo-random confounding neuro-electrical signal to the body to control the respiratory system.

In one embodiment, the transmitted confounding neuro-electrical signal has a frequency in the range of approximately 0.5-4 KHz.

In another embodiment, the transmitted confounding neuro-electrical signal has a frequency in the range of approximately 1-2 KHz.

In another embodiment, the frequency of the confounding neuro-electrical signal is randomly varied.

In another embodiment, the frequency of the confounding neuro-electrical signal is pseudo-randomly varied.

According to the invention, the generated confounding neuro-electrical signals are transmitted to the subject's nervous system.

Preferably, the confounding neuro-electrical signals of the invention are transmitted to the vagus nerve in a multi-directional mode. In one embodiment of the invention, the confounding neuro-electrical signals are transmitted to the vagus nerve via one or more unipolar electrodes that surround the vagus nerve fascicle to stimulate without regard to direction of propagation.

According to the invention, the applied voltage of the confounding neuro-electrical signals can be up to 20 volts to allow for voltage loss during the transmission of the signals. Preferably, current is maintained to less than 2 amp output.

Direct conduction into the nerves via electrodes connected directly to such nerves preferably have outputs less than 3 volts and current less than one tenth of an amp.

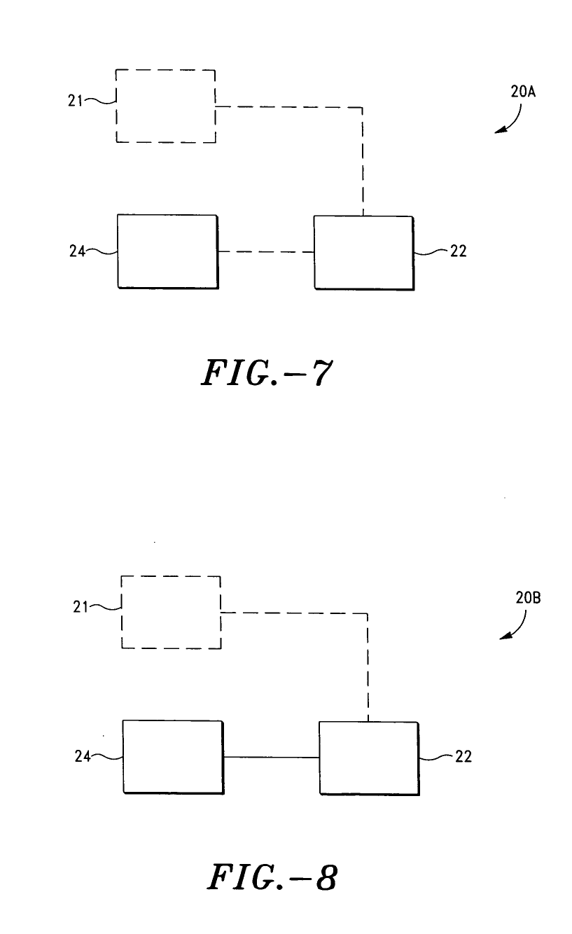

Referring now to FIG. 7, there is shown a schematic illustration of one embodiment of a respiratory control system 20A of the invention. As illustrated in FIG. 7, the control system 20A includes a control module 22, which is adapted to receive coded neurosignals or “waveform signals” from a signal sensor (shown in phantom and designated 21) that is in communication with a subject, and at least one treatment member 24.

The control module 22 is further adapted to generate simulated action potential signals and confounding neuro-electrical signals, and transmit the confounding neuro-electrical signals to the treatment member 24. In some embodiments of the invention, the control module 22 is also adapted to transmit the confounding neuro-electrical signals to the treatment member 24 and, hence, subject (or patient) manually, i.e. upon activation of a manual switch 25.

The treatment member 24 is adapted to communicate with the body and receives the confounding neuro-electrical signal(s) from the control module 22. According to the invention, the treatment member 24 can comprise an electrode, antenna, a seismic transducer, or any other suitable form of conduction attachment for transmitting respiratory neuro-electrical signals that regulate or modulate breathing function in human or animals.

The treatment member 24 can be attached to appropriate nerves or respiratory organ(s) via a surgical process. Such surgery can, for example, be accomplished with “key-hole” entrance in a thoracic-stereo-scope procedure. If necessary, a more expansive thoracotomy approach can be employed for more proper placement of the treatment member 24.

Further, if necessary, the treatment member 24 can be inserted into a body cavity, such as the nose or mouth, and can be positioned to pierce the mucinous or other membranes, whereby the member 24 is placed in close proximity to the medulla oblongata and/or pons. The confounding neuro-electrical signals of the invention can then be sent into nerves that are in close proximity with the brain stem.

Further, if necessary, the treatment member 24 can be inserted in a position underlying the carotid artery in the neck, whereby the member 24 is placed in close proximity to the vagus nerve. The confounding neuro-electrical signals of the invention can then be coupled into the vagus nerve.

As illustrated in FIG. 7, the control module 22 and treatment member 24 can be entirely separate elements, which allow system 20A to be operated remotely. According to the invention, the control module 22 can be unique, i.e., tailored to a specific operation and/or subject, or can comprise a conventional device.

Referring now to FIG. 8, there is shown a further embodiment of a control system 20B of the invention. As illustrated in FIG. 8, the system 20B is similar to system 20A shown in FIG. 7. However, in this embodiment, the control module 22 and treatment member 24 are connected.

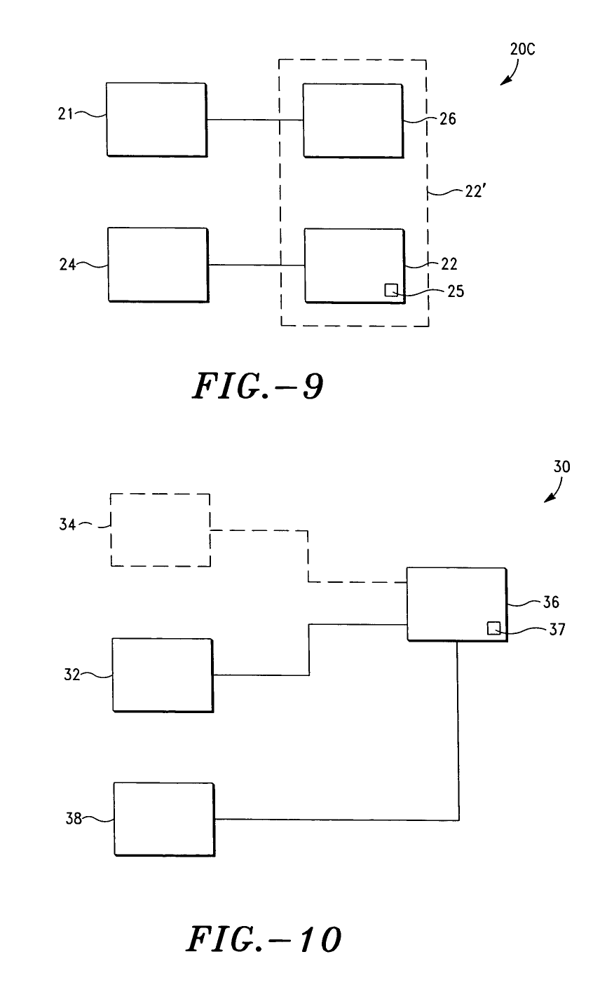

Referring now to FIG. 9, there is shown yet another embodiment of a control system 20C of the invention. As illustrated in FIG. 9, the control system 20C similarly includes a control module 22 and a treatment member 24. The system 20C further includes at least one signal sensor 21.

The system 20C also includes a processing module (or computer) 26. According to the invention, the processing module 26 can be a separate component or can be a sub-system of a control module 22′, as shown in phantom.

As indicated above, the processing module (or control module) preferably includes storage means adapted to store the captured neurosignals or respiratory signals. In a preferred embodiment, the processing module 26 is further adapted to extract and store the components of the captured neurosignals in the storage means according to the function performed by the signal components.

Referring now to FIG. 10, there is shown a further embodiment of a respiratory control system 30. As illustrated in FIG. 10, the system 30 includes at least one respiration sensor 32 that is adapted to monitor the respiration status of a subject and transmit at least one signal indicative of the respiratory status.

According to the invention, the respiration status (and, hence, a respiratory disorder) can be determined by a multitude of factors, including diaphragm movement, respiration rate, levels of O₂ and/or CO₂ in the blood, muscle tension in the neck, air passage (or lack thereof) in the air passages of the throat or lungs, i.e., ventilation. Various sensors can thus be employed within the scope of the invention to detect the noted factors and, hence, the onset of a respiratory disorder.

The system 30 further includes a processor 36, which is adapted to receive the respiratory system status signal(s) from the respiratory sensor 32. The processor 36 is also adapted to receive coded neurosignals recorded by a respiratory signal probe (shown in phantom and designated 34).

The processor 36 is further adapted to generate simulated action potential signals and confounding neuro-electrical signals, and transmit the confounding neuro-electrical signals to the treatment member or transmitter 38. The processor 36 is also adapted to transmit the generated confounding neuro-electrical signals to the transmitter 38 and, hence, patient manually, i.e. upon activation of a manual switch 37.

In a preferred embodiment of the invention, the processor 36 includes storage means for storing the captured neurosignals, respiratory system status signals, and generated simulated action potential and confounding neuro-electrical signals. The processor 36 is further adapted to extract the components of the captured neurosignals and store the signal components in the storage means.

In a preferred embodiment, the processor 36 is programmed to detect respiratory system status signals indicative of respiration abnormalities and/or neurosignals and/or segments or components thereof that are indicative of respiratory system distress and generate at least one simulated action potential signal and/or a confounding neuro-electrical signal.

Referring to FIG. 10, the confounding neuro-electrical signal is routed to a transmitter 38 that is adapted to be in communication with the subject's body. The transmitter 38 is adapted to transmit the confounding neuro-electrical signal(s) to the subject's body (in a similar manner as described above) to control and, preferably, remedy the detected respiration abnormality.

According to the invention, the confounding neuro-electrical signal is preferably transmitted to (i) the phrenic nerve to contract the diaphragm, (ii) the hypoglossal nerve to tighten the throat muscles and/or (iii) the vagus nerve to suppress or mask abnormal respiratory signals, e.g., parasympathetic action potentials that induce bronchial constriction. As indicated, a single confounding neuro-electrical signal or a plurality of confounding neuro-electrical signals (i.e. signal train) can be transmitted in conjunction with one another.

According to the invention, in one embodiment of the invention, the method for controlling respiration in a subject thus includes the steps of (i) generating a confounding neuro-electrical signal, (ii) monitoring the respiration status of the subject and providing at least one respiratory system status signal in response to an abnormal function of the respiratory system, and (iii) transmitting the confounding neuro-electrical signal to the body in response to a respiratory status signal that is indicative of respiratory distress or a respiratory abnormality.

According to the invention, the control of respiration can, in some instances, require sending confounding neuro-electrical signals into one or more nerves, including up to five nerves simultaneously, to control respiration. For example, the correction of asthma or other breathing impairment or disease involves the rhythmic operation of the diaphragm and/or the intercostal muscles to inspire and expire air for the extraction of oxygen and the dumping of waste gaseous compounds, such as carbon dioxide.

As discussed above, a primary symptom of asthma is the constriction of the airways. The airway constriction is due, in significant part, to the contraction of smooth muscle tissue lining the bronchi and bronchioles.

In most instances, the noted airway constriction is induced or exacerbated by abnormal respiratory signals, e.g., parasympathetic action potentials. The abnormal respiratory signal can, however, be suppressed or masked to abate the airway constriction by transmitting a confounding neuro-electrical signal of the invention.

A further symptom of asthma is excessive mucus production. Mucus production, if excessive, can form mucoid plugs that restrict air volume flow throughout the bronchi.

The noted mucus production can, however, also be effectively abated by transmission of the confounding neuro-electrical signals of the invention.

It is also recognized that proinflammatory cytokines can, and in many instances will, contribute to various deleterious characteristics, including airway inflammation, through their release during an inflammatory cytokine cascade. Since mammals respond to inflammation caused by inflammatory cytokine cascades, in part, through central nervous system regulation, it is believed that the confounding neuro-electrical signals of the invention can inhibit and/or reduce proinflammatory cytokine levels in a subject (or patient) when the noted signals are transmitted thereto.

Thus, in accordance with one embodiment of the invention, there is provided a method of inhibiting the release of a proinflammatory cytokine that includes the steps of (i) generating a confounding neuro-electrical signal, and (ii) transmitting the confounding neuro-electrical signal to the body, whereby the secretion of the proinflammatory cytokine is abated.

As will be appreciated by one having ordinary skill in the art, the confounding neuro-electrical signals of the invention can thus be effectively employed to mitigate various symptoms of asthma.

### EXAMPLES

The following examples are provided to enable those skilled in the art to more clearly understand and practice the present invention. They should not be considered as limiting the scope of the invention, but merely as being illustrated as representative thereof.

In each example herein, the swine are challenged with nebulized methacholine, a drug routinely administered in the diagnosis of severity of airway reactivity (reflex broncho-constriction) in asthmatic patients. This is evidenced as airway hyper-reactivity or broncho-constriction that is present in acute asthma attacks and in mid-stage COPD (chronic obstructive pulmonary disease).

### Example 1

A juvenile swine having a weight of 82 lbs was exposed to nebulized methacholine that was dissolved in saline. Ventilation parameters, arterial oxygen saturation and exhaled carbon dioxide were monitored at various concentrations of methacholine.

The vagus nerve of the swine was exposed in the neck. As reflected in Table I, two signals were transmitted to the animal. Signal 1 comprised a sinusoidal signal having an amplitude of ±800 mV. Signal 2 comprised a confounding neuro-electrical signal having a plurality of simulated action potential signals. Each simulated action potential signal had a 200 μsec, 800 mV positive voltage region and a 400 μsec, −400 mV negative voltage region.

TABLE I

| | Positive Amplitude | | Negative Amplitude | | |
|---|---|---|---|---|---|
| | Region | | Region | | |
| Signal | Amplitude | Time | Amplitude | Time | Frequency |
| #1 | 800 mV | | −800 mV | | 500 Hz |
| #2 | 800 mV | 200 μsec | −400 mV | 400 μsec | 1666 Hz |

The animal was administered four different doses of methacholine plus saline; allowed to recover for approximately 30 minutes; then challenged with the third dose of methacholine four more times while transmitting the noted signals.

Referring now to Table II, there is shown a summary of the effects of the methacholine challenge and transmitted signals on selected parameters of respiratory function in the swine.

TABLE II

| | Saline | Methacholine | | |
|---|---|---|---|---|
| Parameter | Alone | Alone | Signal 1 | Signal 2 |
| Tidal Volume | 377 | 509 | 452 | 261 |
| (mL) | | | | |
| Respiration | 25 | 16 | 15 | 11 |
| Rate | | | | |
| (BPM) | | | | |
| Inspiratory | 0.36 | 0.93 | 0.60 | 0.27 |
| Pressure | | | | |
| (cmH₂O) | | | | |
| O₂ Saturation | 88 | 81 | 78 | 80 |
| (%) | | | | |
| Manual | 0 | 21 | 105 | 0 |
| Ventilation | | | | |
| required | | | | |
| (sec) | | | | |

Upon administration of methacholine and transmittal of signal #1 the swine went into respiratory arrest sooner (as compared to the baseline administration of saline alone) and, as reflected in Table II, had to be manually ventilated for approximately two minutes to recover, i.e. breathe on its own. Upon administration of methacholine and transmittal of signal #3, the swine responded as though it were inhaling nebulized saline, i.e. did not go into respiratory arrest during the three minute methacholine challenge. Signal #2 thus confounded the normal broncho-constrictive reflex.

Table II further reflects that, upon administration of methacholine and transmittal of signal #2, there was a marked reduction in respiratory rate and effort, which were similar to baseline levels without administration of methacholine.

Example 1 thus reflects that a confounding neuro-electrical signal of the invention mitigates the adverse effects of a broncho-constrictive pharmacologic agent and that other neuro-active signals compound such adverse effects.

### Example 2

A juvenile swine weighing approximately 70 lbs was prepared for surgery and then challenged with nebulized solutions of saline having increasing concentrations of methacholine. The challenges lasted three minutes with a seven minute rest period between challenges.

The swine went into respiratory arrest after 1:20 minutes when a dose of 2 mg/ml of methacholine was administered. After manual ventilation, the swine recovered and began spontaneous breathing. This dose was administered repeatedly while the effect of signal amplitude was investigated.

In the next phase of the study, electrodes were inserted into each vagus nerve and four confounding neuro-electrical signals were transmitted to the swine. Signal #1 comprised a confounding neuro-electrical signal having a plurality of simulated action potential signals having a 200 μsec, 1500 mV positive voltage region and a 400 μsec, −750 mV negative voltage region. Signal #2 comprised a confounding neuro-electrical signal having a plurality of simulated action potential signals having a 200 μsec, 1800 mV positive voltage region and a 400 μsec, −900 mV negative voltage region. Signal #3 comprised a confounding neuro-electrical signal having a plurality of simulated action potential signals having a 300 μsec, 1500 mV positive voltage region and a 600 μsec, −750 mV negative voltage region. Signal #4 comprised a confounding neuro-electrical signal having a plurality of simulated action potential signals having a 300 μsec, 1800 mV positive voltage region and a 600 μsec, −900 mV negative voltage region.

The noted signals are set forth in Table III.

TABLE III

| | Positive Amplitude | | Negative Amplitude | | |
|---|---|---|---|---|---|
| | Region | | Region | | |
| Signal | Amplitude | Time | Amplitude | Time | Frequency |
| #1 | 1500 mV | 200 μsec | −750 mV | 400 μsec | 1666 Hz |
| #2 | 1800 mV | 200 μsec | −900 mV | 400 μsec | 1666 Hz |
| #3 | 1500 mV | 300 μsec | −750 mV | 600 μsec | 1100 Hz |
| #4 | 1800 mV | 300 μsec | −900 mV | 600 μsec | 1100 Hz |

Referring now to Table IV, there is shown a summary of the effects of the methacholine challenge and transmitted signals on survival time before requiring manual ventilation.

TABLE IV

| | Time to respiratory arrest |
|---|---|
| Signal | (min) |
| Methacholine | 1:20 |
| alone | |
| #1 | 1:50 |
| #2 | 2:00 |
| #3 | 2:00 |
| #4 | 2:00 |

As reflected in Table IV, upon administration of Signals 1-4, the animal was able to survive the methacholine challenge for a greater period of time than when the same dose of methacholine was administered without a confounding neuro-electrical signal. In addition, the effectiveness of the frequency range is demonstrated at both 1100 Hz and 1666 Hz.

It is important to note that in prior studies, the survival time of the animal decreased with additional challenges with the same methacholine dose. The confounding neuro-electrical signals produced increasing survival time compared to the baseline level in spite of repeated challenges that produced respiratory arrest.

### Example 3

A juvenile swine weighing 44 lbs. was exposed to nebulized methacholine that was dissolved in saline. The dose of methacholine was titrated to a level which induced respiratory arrest in approximately 1 minute.

The animal was manually ventilated and allowed to recover. After the animal recovered, the same dose of methacholine was administered with a confounding neuro-electrical signal. As reflected in Table V, two different confounding neuro-electrical signals were transmitted to the animal. Signal #1 comprised a confounding neuro-electrical signal having a plurality of simulated action potential signals having a positive amplitude of approximately 1500 mV for a duration of 300 μsec and a negative amplitude of approximately −750 mV for a duration of 600 μsec. Signal #2 comprised a confounding neuro-electrical signal having a plurality of simulated action potential signals having a positive amplitude of approximately 1200 mV for a duration of 300 μsec and a negative amplitude of approximately −600 mV for a duration of 600 μsec. Each of the noted confounding neuro-electrical signals had a frequency of approximately 1111 Hz.

TABLE V

| | Positive Amplitude | | Negative Amplitude | | |
|---|---|---|---|---|---|
| | Region | | Region | | |
| Signal | Amplitude | Time | Amplitude | Time | Frequency |
| #1 | 1500 mV | 300 μsec | −750 mV | 600 μsec | 1111 Hz |
| #2 | 1200 mV | 300 μsec | −600 mV | 600 μsec | 1111 Hz |

When the confounding neuro-electrical signals were administered bilaterally to the vagus nerve of the swine for 45 seconds prior to the administration of the methacholine, the animal was able to survive the entire 3 minute challenge without respiratory arrest.

### Example 4

A juvenile swine, weighing 60 lbs., was similarly exposed to nebulized methacholine that was dissolved in saline. The dose of methacholine was titrated to a level which induced severe respiratory distress within 3 minutes. Next, stimulus was applied bi-laterally to the vagus nerve until a level was reached that produced a sustained observable effect on spontaneous ventilation.

The confounding neuro-electrical signal comprised a plurality of simulated action potentials having a positive amplitude of 2.0 V for a duration of 300 μsec and a negative amplitude of −1.0 V for a duration of 600 μsec. The confounding neuro-electrical signal had a frequency of approximately 1111 Hz.

The same dose of methacholine was administered with the confounding neuro-electrical signal.

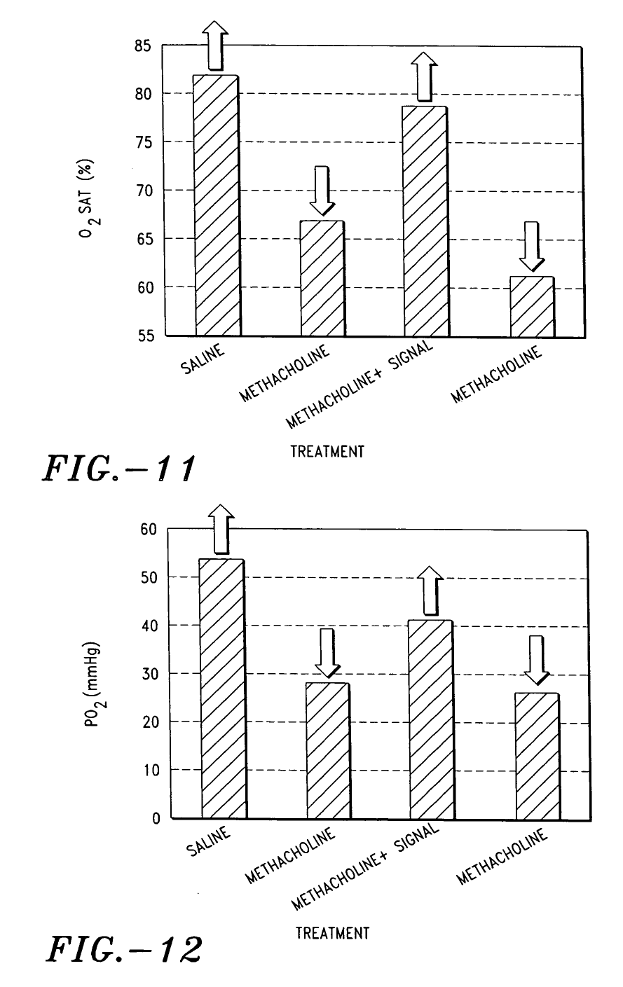

Referring now to FIG. 11, there is shown the effects of the methacholine challenge and transmitted signal on arterial oxygen during a 3 minute methacholine challenge at 15 mg/ml concentration.

It can be seen that oxygen saturation with the confounding signal present is significantly greater than when the confounding signal is not present, i.e. 79% when present as compared to 61 to 67% when confounding signal is not present.

Referring now to FIG. 12, there is shown the effects of the methacholine challenge and transmitted signal on partial pressure of arterial oxygen during a 3 minute methacholine challenge at 15 mg/ml concentration.

It can be seen that partial pressure of arterial oxygen with the confounding signal present is significantly greater than when the confounding signal is not present, i.e. 41 mm Hg when present as compared to 26 to 28 mm Hg when confounding signal is not present.

As will be appreciated by one having ordinary skill in the art, the confounding neuro-electrical signals of the invention can thus be effectively employed to mitigate the normal human response to asthma triggers, reduce the severity of asthma attacks and permit delivery of anti-inflammatory medication for better control of asthma symptoms during acute attacks.

In a recent study by Assignee, Science Medicus, Inc., respiratory neurosignals were acquired from the phrenic nerve of a rat and stored in a processor memory (as described herein). The neurosignals were subsequently transmitted to a dog (i.e. beagle) without added voltage, current or modification, whereby control of the dog's diaphragm muscles and, hence, respiratory function was effectuated. The noted study thus established that neurosignal (and neuro-code) similarity exists between various, and most likely all, common mammalian species.

Thus, while the simulated action potential frequencies and amplitudes of the confounding neuro-electrical signals employed in the above listed examples have been demonstrated to be effective on domestic swine, it is reasonable to conclude that the simulated action potential frequencies and amplitudes and, hence, confounding neuro-electrical signals embodying same, would not be substantially different in other mammalian species, including humans. Determination of effective confounding neuro-electrical signals for a particular species would thus not require undue experimentation for one skilled in the art.

The present invention thus provides methods and apparatus to effectively control respiration and abate numerous respiratory abnormalities. As indicated above, a primary symptom of asthma is the constriction of the airways. The airway constriction is due, in significant part, to the contraction of smooth muscle tissue lining the bronchi and bronchioles, which is induced or exacerbated by abnormal respiratory signals, e.g., parasympathetic action potentials. By transmitting a confounding neuro-electrical signal of the invention the abnormal respiratory signal can be suppressed or masked to abate the airway constriction.

A further symptom of asthma is excessive mucus production. Mucus production, if excessive, can form mucoid plugs that restrict air volume flow throughout the bronchi.

The noted mucus production can, however, be effectively abated by transmission of the confounding neuro-electrical signals of the invention.

Further, by controlling bronchial constriction and mucinous action in the bronchi, chronic airway obstructive disorders, such as emphysema, can also be addressed. The ability to control bronchial constriction will also be useful for emergency room treatment of acute bronchitis or smoke inhalation injuries.

Acute fire or chemical inhalation injury treatment can also be enhanced through the methods and apparatus of the invention, while using mechanical respiration support. Injury-mediated mucus secretions also lead to obstruction of the airways and are refractory to urgent treatment, posing a life-threatening risk. Edema (swelling) inside the trachea or bronchial tubes tends to limit bore size and cause oxygen starvation.

The breathing effort of patients with pneumonia may also be eased by modulated activation of the phrenic nerve through the methods and apparatus of the invention.

As will be appreciated by one having ordinary skill in the art, the confounding neuro-electrical signals of the invention can also be employed to suppress other (non-respiratory related) neurosignals and/or action potentials that induce abnormal or undesirable organ or system function. Indeed, it is well known that virtually all action potentials that are naturally generated in the body are similar in form and, hence, subject to suppression by the confounding neuro-electrical signals of the invention.

Thus, the confounding neuro-electrical signals of the invention can be employed, for example, to abate neuro-electrical signals or action potential signals that are associated with pain, autonomic dysreflexia, shock, hypertension, or other neurogenic reflexive disorders.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for suppressing a reflex action in a mammalian body, comprising the steps of:
generating a confounding neuro-electrical signal that is adapted to suppress at least one interneuron that induces the reflex action in the body; and
transmitting said confounding neuro-electrical signal to the body.

2. The method of claim 1, wherein said confounding neuro-electrical signal is transmitted to the nervous system in the body.

3. The method of claim 2, wherein said confounding neuro-electrical signal is transmitted to the vagus nerve.

4. The method of claim 1, wherein said confounding neuro-electrical signal is adapted to suppress at least one parasympathetic action potential that induces said reflex action.

5. The method of claim 1, wherein said reflex action comprises bronchial constriction.

6. The method of claim 1, wherein said confounding neuro-electrical signal includes a plurality of simulated action potential signals, each of said plurality of simulated action potential signals having a first region having a positive amplitude in the range of approximately 100-2000 mV for a first period of time in the range of approximately 100-400 μsec and a second region having a negative amplitude in the range of approximately −50 mV to −1000 mV for a second period of time in the range of approximately 200-800 μsec.

7. The method of claim 6, wherein said confounding neuro-electrical signal has a frequency in the range of approximately 1-2 KHz.

8. A method for controlling respiration in a subject, comprising the steps of:
generating a confounding neuro-electrical signal that is adapted to suppress at least one interneuron that induces a respiratory reflex action in the subject's body; and
transmitting said confounding neuro-electrical signal to the nervous system of the subject.

9. The method of claim 8, wherein said confounding neuro-electrical signal is transmitted to the vagus nerve.

10. The method of claim 8, wherein said confounding neuro-electrical signal is adapted to suppress at least one parasympathetic action potential that induces said respiratory reflex action.

11. The method of claim 8, wherein said respiratory reflex action comprises bronchial constriction.

12. The method of claim 8, wherein said confounding neuro-electrical signal includes a plurality of simulated action potential signals, each of said plurality of simulated action potential signals having a first region having a positive amplitude in the range of approximately 100-2000 mV for a first period of time in the range of approximately 100-400 μsec and a second region having a negative amplitude in the range of approximately −50 mV to −1000 mV for a second period of time in the range of approximately 200-800 μsec.

13. The method of claim 12, wherein said confounding neuro-electrical signal has a frequency in the range of approximately 1-2 KHz.

14. A method for treating a pathophysiology of asthma in a subject, comprising the steps of:
generating a confounding neuro-electrical signal that is adapted to suppress at least one abnormal respiratory signal that induces a pathophysiology of asthma; and
transmitting said confounding neuro-electrical signal to the nervous system of the subject.

15. The method of claim 14, wherein said confounding neuro-electrical signal is transmitted to the vagus nerve.

16. The method of claim 14, wherein said pathophysiology of asthma comprises a pathophysiology selected from the group consisting of bronchial hyper-responsiveness, smooth muscle hypertrophy, mucus hyper-secretion and hyper-secretion of a proinflammatory cytokine.

17. The method of claim 14, wherein said confounding neuro-electrical signal has a first region having a positive amplitude in the range of approximately 100-2000 mV for a first period of time in the range of approximately 100-400 μsec and a second region having a negative amplitude in the range of approximately −50 mV to −1000 mV for a second period of time in the range of approximately 200-800 μsec.

18. The method of claim 17, wherein said confounding neuro-electrical signal has a frequency in the range of approximately 1-2 KHz.

19. A method for treating bronchial constriction of a subject, comprising the steps of:
generating a confounding neuro-electrical signal that is adapted to suppress at least one group of reflex mediating interneurons that induces bronchial constriction; and
transmitting said confounding neuro-electrical signal to the nervous system of the subject, whereby said bronchial constriction is abated.

20. The method of claim 19, wherein said confounding neuro-electrical signal is transmitted to the vagus nerve.

21. The method of claim 21, wherein said confounding neuro-electrical signal includes a plurality of simulated action potential signals, each of said simulated action potential signals having a first region having a positive amplitude in the range of approximately 100-2000 mV for a first period of time in the range of approximately 100-400 μsec and a second region having a negative amplitude in the range of approximately −50 mV to −1000 mV for a second period of time in the range of approximately 200-800 μsec.

22. The method of claim 21, wherein said confounding neuro-electrical signal has a frequency in the range of approximately 1-2 KHz.

23. A method for controlling respiration in a subject, comprising the steps of:
generating a simulated action potential signal that is recognizable by the respiration system as a modulation signal, said simulated action potential having a first region having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec;
generating a confounding neuro-electrical signal, said confounding neuro-electrical signal including a plurality of said simulated action potential signals; and
transmitting said confounding neuro-electrical signal to the nervous system of the subject.

24. The method of claim 23, wherein said confounding neuro-electrical signal is transmitted to the subject's vagus nerve.

25. The method of claim 23, wherein said confounding neuro-electrical signal has a frequency in the range of approximately 1-2 KHz.

26. A method for controlling respiration in a subject, comprising the steps of:
generating a random confounding neuro-electrical signal, said random confounding neuro-electrical including a plurality of random simulated action potential signals, each of said random simulated action potential signals having a first region having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec; and
transmitting said random confounding neuro-electrical signal to the nervous system of the subject.

27. The method of claim 29, wherein said random confounding neuro-electrical signal is transmitted to the subject's vagus nerve.

28. The method of claim 26, wherein said random confounding neuro-electrical signal has a frequency in the range of approximately 1-2 KHz.

29. The method of claim 28, wherein said frequency is randomly varied.

30. The method of claim 29, wherein said frequency is randomly varied between approximately 40-4000 Hz.

31. The method of claim 26, wherein said first region of said random confounding neuro-electrical signal is randomly varied.

32. The method of claim 26, wherein the normalized positive amplitude of said random confounding neuro-electrical signal is randomly varied between approximately 0.95-1.05 times the average positive amplitude.

33. The method of claim 26, wherein said second region of said random confounding neuro-electrical signal is randomly varied.

34. The method of claim 26, wherein the normalized negative amplitude of said random confounding neuro-electrical signal is randomly varied between approximately 0.95-1.05 times the average negative amplitude.

35. The method of claim 26, wherein said first period of time of said random confounding neuro-electrical signal is randomly varied.

36. The method of claim 35, wherein said first period of time is randomly varied between approximately 0.25-5.0 milliseconds.

37. The method of claim 26, wherein said second period of time of said random confounding neuro-electrical signal is randomly varied.

38. The method of claim 37, wherein said second period of time is randomly varied between approximately 0.25-5.0 milliseconds.

39. The method of claim 26, wherein said random confounding neuro-electrical signal comprises a signal train having a plurality of said random confounding neuro-electrical signals with randomly varied intervals therebetween.

40. The method of claim 39, wherein said intervals between said random confounding neuro-electrical signals is randomly varied between approximately 0.5-1.0 millisecond.

41. A method for controlling respiration in a subject, comprising the steps of:
generating a random confounding neuro-electrical signal, said random confounding neuro-electrical including a plurality of random simulated action potential signals, each of said random simulated action potential signals having a first region having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec;
monitoring the respiration status of the subject and providing at least one respiratory system status signal in response to an abnormal function of the respiratory system; and
transmitting said random confounding neuro-electrical signal to the nervous system of the subject in response to a respiratory status signal that is indicative of a respiratory abnormality.

42. The method of claim 41, wherein said random confounding neuro-electrical signal is transmitted to the subject's vagus nerve.

43. The method of claim 41, wherein said random confounding neuro-electrical signal has a frequency in the range of approximately 1-2 KHz.

44. The method of claim 43, wherein said frequency is randomly varied.

45. The method of claim 44, wherein said frequency is randomly varied between approximately 40-4000 Hz.

46. The method of claim 41, wherein said positive amplitude of said random confounding neuro-electrical signal is randomly varied.

47. The method of claim 41, wherein said negative amplitude of said random confounding neuro-electrical signal is randomly varied.

48. The method of claim 41, wherein said first period of time of said random confounding neuro-electrical signal is randomly varied.

49. The method of claim 41, wherein said second period of time of said random confounding neuro-electrical signal is randomly varied.

50. A method for controlling respiration in a subject, comprising the steps of:
generating a pseudo-random confounding neuro-electrical signal, said pseudo-random confounding neuro-electrical including a plurality of pseudo-random simulated action potential signals, each of said pseudo-random simulated action potential signals having a first region having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec; and
transmitting said pseudo-random confounding neuro-electrical signal to the nervous system of the subject.

51. The method of claim 50, wherein said pseudo-random confounding neuro-electrical signal is transmitted to the subject's vagus nerve.

52. The method of claim 50, wherein said pseudo-random confounding neuro-electrical signal has a frequency in the range of approximately 1-2 KHz.

53. The method of claim 52, wherein said frequency is pseudo-randomly varied.

54. The method of claim 53, wherein said frequency is pseudo-randomly varied between approximately 40-4000 Hz.

55. The method of claim 50, wherein said first region of said pseudo-random confounding neuro-electrical signal is pseudo-randomly varied.

56. The method of claim 50, wherein the normalized positive amplitude of said pseudo-random confounding neuro-electrical signal is pseudo-randomly varied between approximately 0.95-1.05 times the average positive amplitude.

57. The method of claim 50, wherein said second region of said pseudo-random confounding neuro-electrical signal is pseudo-randomly varied.

58. The method of claim 50, wherein the normalized negative amplitude of said first pseudo-random confounding neuro-electrical signal is pseudo-randomly varied between approximately 0.95-1.05 times the average negative amplitude.

59. The method of claim 50, wherein said first period of time of said pseudo-random confounding neuro-electrical signal is pseudo-randomly varied.

60. The method of claim 59, wherein said first period of time is pseudo-randomly varied between approximately 0.25-5.0 milliseconds.

61. The method of claim 50, wherein said second period of time of said pseudo-random confounding neuro-electrical signal is pseudo-randomly varied.

62. The method of claim 61, wherein said second period of time is pseudo-randomly varied between approximately 0.25-5.0 milliseconds.

63. The method of claim 50, wherein said pseudo-random confounding neuro-electrical signal comprises a signal train having a plurality of said pseudo-random confounding neuro-electrical signals with pseudo-randomly varied intervals therebetween.

64. The method of claim 63, wherein said intervals between said pseudo-random confounding neuro-electrical signals is pseudo-randomly varied between approximately 0.5 -1 millisecond.

65. A method for controlling respiration in a subject, comprising the steps of:
generating a pseudo-random confounding neuro-electrical signal, said pseudo-random confounding neuro-electrical including a plurality of pseudo-random simulated action potential signals, each of said pseudo-random simulated action potential signals having a first region having a positive amplitude in the range of approximately 100 to 2000 mV for a first period of time in the range of approximately 100-400 μsec and a second region having a negative amplitude in the range of approximately −50 mV to −1000 mV for a second period of time in the range of approximately 200-800 μsec;
monitoring the respiration status of the subject and providing at least one respiratory system status signal in response to an abnormal function of the respiratory system; and
transmitting said pseudo-random confounding neuro-electrical signal to the nervous system of the subject in response to a respiratory status signal that is indicative of a respiratory abnormality.

66. The method of claim 65, wherein said pseudo-random confounding neuro-electrical signal is transmitted to the subject's vagus nerve.

67. The method of claim 65, wherein said pseudo-random confounding neuro-electrical signal has a frequency in the range of approximately 1-2 KHz.

68. The method of claim 67, wherein said frequency is pseudo-randomly varied.

69. The method of claim 68, wherein said frequency is pseudo-randomly varied between approximately 40-4000 Hz.

70. The method of claim 65, wherein said positive amplitude of said pseudo-random confounding neuro-electrical, signal is pseudo-randomly varied.

71. The method of claim 65, wherein said negative amplitude of said pseudo-random confounding neuro-electrical signal is pseudo-randomly varied.

72. The method of claim 65, wherein said first period of time of said pseudo-random confounding neuro-electrical signal is pseudo-randomly varied.

73. The method of claim 65, wherein said second period of time of said pseudo-random confounding neuro-electrical signal is pseudo-randomly varied.

74. A confounding neuro-electrical signal having a plurality of simulated action potential signals, each of said simulated action potential signals having a first region having a first positive amplitude in the range of approximately 100-2000 mV for a first period of time in the range of approximately 100-400 μsec, a second region having a first negative amplitude in the range of approximately −50 mV to −1000 mV for a second period of time in the range of approximately 200-800 μsec and a frequency in the range of approximately 1-2 KHz, said confounding neuro-electrical signal being adapted to suppress at least one interneuron that induces a reflex action in the body when transmitted thereto.

75. The confounding neuro-electrical signal of claim 74, wherein said confounding neuro-electrical signal is adapted to confound at least one parasympathetic action potential that induces said reflex action.

76. The method of claim 74, wherein said reflex action comprises a respiratory reflex action.

77. The method of claim 76, wherein said respiratory reflex action comprises bronchial constriction.
